(19) United States
(12) Patent Application Publication
(10) Pub. No.: US 2022/0223021 A1
(43) Pub. Date: Jul. 14, 2022

(54) SYSTEMS AND METHODS FOR REMOTELY MONITORING A WEARABLE DEVICE USED FOR SENIOR CARE

(71) Applicant: VieConnect US, Phoenix, AZ (US)

(72) Inventors: Dimitri Tintikakis, Toulouse (FR); Sotirios Koufos, Thessaloniki (GR); Yevgeniy Nurseitov, Toulouse (FR); Guillermo Romero, Phoenix, AZ (US)

(21) Appl. No.: 17/260,888

(22) PCT Filed: Jan. 14, 2021
(86) PCT No.: PCT/US21/13435

§ 371 Date: Jan. 15, 2021

(51) Int. Cl. G08B 21/02; A61B 5/11; A61B 5/00

(52) U.S. Cl. CPC G08B 21/02; A61B 5/1116; A61B 2560/0242; A61B 2503/08; A61B 5/0022

(57) ABSTRACT

A wetness or saturation and temperature, and motion, and posture or orientation detecting distributed system that can include a wireless module, a wireless hub and a local or cloud server. A distributed algorithm that is stored and executed in the wireless module and the wireless hub or cloud or local server can detect wetness or saturation of the undergarment and temperature, and motion, and posture or orientation of the wearer and can also automatically count the number of undergarment changes performed daily by the caregivers.

### CROSS REFERENCE TO RELATED APPLICATIONS

This application claims the benefit under 35 USC § 365(c) of International Patent Application No. PCT/US21/13435 filed on Jan. 14, 2021. The International Patent Application No. PCT/US21/13435 is hereby incorporated by reference in its entirety.

### BACKGROUND OF THE INVENTION

### Field of the Invention

The present invention relates to an environmental sensing device and more particularly to a remote monitoring system for indicating the need for an article of clothing to be changed when saturated or soiled.

### Description of Related Art

In nursing homes, mental institutions, care centers, or other caregiving facilities an individuals' incontinent undergarment is usually checked on every few hours. If the individual releases their urine or other bodily fluid right after being checked this condition can go unnoticed for several hours or more. In addition, a significant percentage of people are not able to effectively communicate episodes of incontinence or loss of bladder control with their caregivers. As a result, many people experience long periods of time in which their clothing or incontinent undergarment is saturated in urine which exposes that individual to harsh and irritating conditions caused by urine and other bodily fluids. A condition commonly known as “diaper rash” or other infections may develop making it even more critical for the caregiver to change the patient's clothing or incontinence undergarment. Moreover, individuals that live in nursing homes are usually woken up every few hours, interrupting their sleep, for the caregiver to check their incontinent undergarment.

Various types of devices for monitoring, detecting and notifying of the condition of a diaper, bedding, or adult incontinence undergarment exist in the art. The general principle of a wetness detection system that is known in the art usually activates an audible or visible alarm when two electrodes complete a circuit when in the presence of urine or other bodily fluids. This usually is accomplished by detecting some sort of threshold of wetness within an incontinence undergarment. Several attempts have been made in the past to address moist incontinence undergarments. For example, Kline et al., U.S. Pat. No. 5,264,830 discloses a sensing device which uses an alarm positioned on the outside of the diaper. The device includes a small battery-powered audible alarm connected to an electrical circuit having contacts that when wet completes a circuit and sounds an alarm. The problem with this device is that the caregiver is not usually in the same room as the individual to hear an audible alarm or see a visual indicator. In addition, the device usually requires the user to wear waterproof pants that adds to the wearer's clothing making he/she uncomfortable when wearing the undergarment.

While these and similar devices have been somewhat successful in signaling an event within an undergarment, they still tend to exhibit numerous problems and shortcoming inherent with the respective designs. Accordingly, a significant advance in the art could be realized if a device could be developed that could monitor, detect, and then alert a caretaker remotely when the individual has soiled their undergarment. The device may be small and compact as to not interfere with or cause any discomfort to the individual, yet should not be readily swallowed or removed from the undergarment. In addition, the device may be sensitive enough to detect the slightest humidity, temperature or orientation change, and then promptly alert the caretaker after detecting saturation, elevated temperature, motion or a posture change event.

Accordingly, there exists a continuing and unaddressed need for a device that can monitor, detect and notify a caretaker or family member when there is an event with an individual's undergarment. In addition, advancements in the art allow the caretaker, or family member to track remotely the initial wetness event from when the undergarment is changed through an app or webpage on a smartphone, or computer web portal.

### SUMMARY OF THE INVENTION

Aspects disclosed herein relates to a system and methods for monitoring, detecting, and notifying an individual when someone has a saturation event in their undergarment or temperature, posture or orientation change of his/her body. A wetness, motion, posture and orientation detecting system can comprise a wireless module with a controller accommodated within a housing at least partially separating the controller from the environment to keep the controller and any power source for the controller from getting wet and shorting. An environmental sensor at least partially in contact with the environment around the wireless module detects at least one feature of the environment such as humidity, temperature, or the presence or absence of a particular compound or molecule. A motion sensor, such as an accelerometer, gyroscope, barometer or magnetometer, detects a motion or an orientation of the module and may relate that motion or orientation to the motion or orientation of the wearer of the wireless module. A transceiver may be included to allow the module to transmit information to and receive information or direction from a remote device, such as a wireless communications computer, a cell phone or other mobile communications device. A memory medium may be used to provide executable instruction to the processing unit and/or to store data collected by the environmental or motion and orientation sensors.

A transceiver may be included to receive data from the processing unit and communicate program instructions to the processing unit and allow the wireless module to transmit information to a wireless hub and receive information or direction from a wireless hub, such as a wireless communications computer, a smartphone or other mobile communications device.

In a particular embodiment, the environmental sensor is within the housing with the controller and the housing has at least one thru hole substantially positioned over the environmental sensors, with a bushing substantially positioned between the environmental sensors and the housing. A filter element and a locking ring may also be provided, wherein the filter element is affixed into the bushing and the locking ring secures the filter element substantially between the housing and the locking ring.

The housing may be sealed by a cover encapsulating the controller and a power supply to ensure that the wireless module is resistant to fluids. The power supply may be a rechargeable or replaceable battery. The controller may be configured to intermittently activate the battery to conserve energy and extend battery life.

The wireless module may be secured into a secondary pouch wherein the secondary pouch can be removably attached to an interior of an undergarment or under a patch wherein the patch can be removably attached to an interior of an undergarment. The secondary pouch may have a plurality of holes on its surface to allow fluids or humidity to penetrate the secondary pouch. The wireless module according to claim 2, wherein the remote device or wireless hub is a wireless communications computer or a cell phone or other mobile communications device.

The wireless module may be used to determine whether there is an environmental change with the environmental sensors, and then transmit that state to a remote hub or wireless hub, local or cloud server or other device.

The objects of the invention are further accomplished by a method of detecting wetness or saturation of an undergarment, and motion, posture or orientation of the wearer through securing a wireless module into a secondary pouch or directly attaching it to an interior of an undergarment, monitoring the environmental conditions of the undergarment and the motion, posture or orientation of the wearer and transmitting data related to the environmental characteristics and the wearer's motion, posture or orientation from the wireless module to remote device or wireless hub where the data are processed and then data and events are communicated to a local or cloud server where they are stored. Alerts can be created based on the data alerting a caregiver when the undergarment becomes wet, saturated or soiled or the wearer of the undergarment is moving, has changed posture or fallen down.

The system may use a distributed architecture of the undergarment wetness or saturation detection algorithm based on high performance neural networks or gradient boosting decision tree models and enabling personalized care as required by the user's incontinence profile. Moreover, the personalized algorithm may detect automatically the removal of a wet or saturated undergarment and insertion of a clean undergarment in replacement and may as a result count the number of undergarment changes performed daily by the caregivers thus providing reliable and traceable information on the quality of the incontinence care.

Historical data such as the last known environmental condition or motion, posture or orientation characteristic may be stored and changes to that environmental condition or motion, posture or orientation will create an alert that is conveyed to a caregiver. For example, an alert may be created when the undergarment changes from dry to wet or saturated or an abnormal motion or a posture change or a fall of the wearer has been detected. The historical data may also be used to keep track of the wearer of the undergarment and frequency of abnormal activity, falls and/or soiling of the undergarment. A display of the current status of the wearer may be accessed by a caregiver at any time.

The alerts or information may be transmitted to the caregiver or other personnel via a mobile communications device such as a smartphone, personal digital assistant, a tablet device, a wearable device, a table PC, a laptop, a smart book, or an ultra-book, or forwarded to a third-party notification or data storage system. Software or other data stored on the wireless module may be updated remotely through a remote device or wireless hub, a local or cloud server, or other remote device.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 illustrates an isometric view of a wetness or saturation and motion, posture and orientation detecting system within an undergarment in accordance to one, or more embodiments;

FIG. 2 illustrates an exploded isometric view of a wetness or saturation and motion, posture and orientation detecting system within an undergarment in accordance to one, or more embodiments;

FIG. 2B illustrates an exploded isometric view of another embodiment of a wetness or saturation and motion, posture and orientation detecting system within an undergarment in accordance to one, or more embodiments;

FIG. 3 illustrates a cross-sectional view of a wetness or saturation and motion, posture and orientation detecting system within an undergarment in accordance to one, or more embodiments;

FIG. 4A illustrates a process flow diagram for depicting a method or system for detecting humidity or saturation by inserting a wireless module into an undergarment in accordance to one, or more embodiments;

FIG. 4B illustrates a process flow diagram for depicting a method or system for detecting humidity or saturation by taping a wireless module into an undergarment in accordance to one, or more embodiments;

FIG. 5 illustrates a process flow diagram 500 depicting a method for detecting the state of the wireless module 100, in accordance with one, or more embodiments;

FIG. 6 illustrates a process flow diagram 600 for depicting a method or system for monitoring, detecting and alerting when humidity or saturation is detected in an undergarment in accordance to one, or more embodiments;

FIG. 7 illustrates a process flow diagram for depicting a method or system for detecting automatically removal of a wet or saturated undergarment and insertion of a clean undergarment in replacement and counting the number of undergarment changes performed daily by the caregivers; and

FIG. 8 illustrates a process flow diagram for depicting a distributed method or system for detecting and alerting automatically the presence of a saturated undergarment.

### DETAILED DESCRIPTION OF THE INVENTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

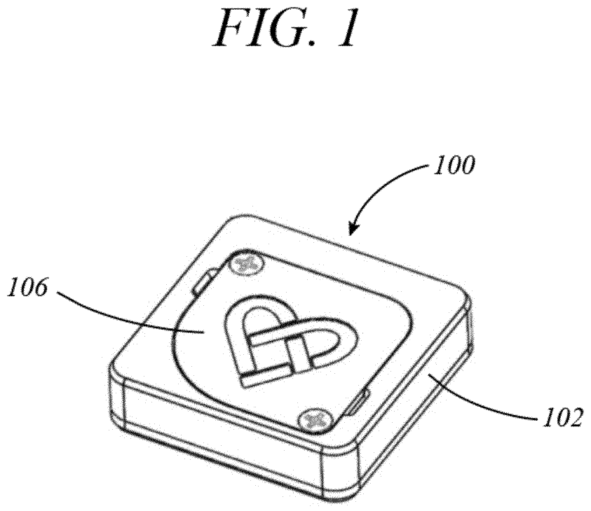

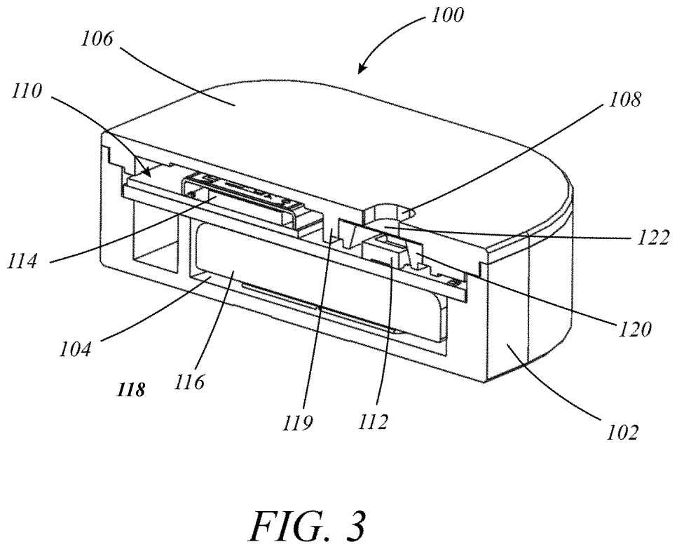

Referring generally to FIG. 1 through 3, which illustrates a wetness or saturation and motion, posture or orientation detecting system in an undergarment, shown generally at 100. In a preferred embodiment, a wireless module 100 can comprise a housing 102, a cover 106, and a controller 110. The controller 110 can comprise environmental sensors 112, a motion sensor 116, and a processing unit. A memory medium may be considered a processing unit. The environmental sensors can be any combination of a temperature sensor, a humidity sensor, a pressure sensor, a gas sensor, or the like that can monitor and detect environmental conditions proximal to the wireless module 100 within the undergarment or other article of clothing. The environmental sensor 112 can be a dual sensor module comprising both a humidity and temperature sensor, or can be a single separate sensor comprising a humidity sensor, and a temperature sensor. In another embodiment, the environmental sensor can detect the presence of a given compound, such as, for example, urea, or another biological compound. The humidity sensor can be, for example, a relative humidity (RH) sensor, absolute humidity (moisture) sensor, or the like. The temperature sensor can be, for example, thermocouple, thermistor, resistance temperature detector, semiconductor based sensor, or the like. The motion sensor 116 can be, but is not limited to a single axis, 2-axis, or a 3-axis digital accelerometer, optical, piezoelectric, capacitive, electromechanical, gyroscope, magnetometer, barometer, or the like. The controller 110 base material can be manufactured out of a single sided, double sided, multilayered, flex, or the like printed circuit board (“PCB”).

In some embodiments, the wireless module 100 can further comprise a processing unit and a wireless communications module which can be, for example, a BLUETOOTH Low Energy (BLE), BLUETOOTH module, Wi-Fi module, IEEE 802.15.4, Z-Wave, Single/Dual Mode Radio Chip or the like. The memory medium can be, for example, random access, flash, read only, or the like, and can store embedded software that provides commands to the processing unit, communication module, environmental sensors, or motion analyzer, and stores and controls the data received from or to be sent to a wireless hub. The processing unit can execute program instructions that can be stored on the memory medium, such as, but not limited to, instructions on monitoring, detecting and transmitting when the undergarment has undergone a wetness or saturation event, and/or instructions on when the orientation of the wearer has changed wherein the orientation can be a fall, and/or battery notification, and/or when the wireless module is removed from and placed into an undergarment, or the like. In some embodiments, the module 100 can be configured to be paired with the intended user of the module 100. A user profile can be created that will be associated with the module 100. The user profile can include the user's age, sex, address or room number, medical condition, or any other information known by those skilled in the art.

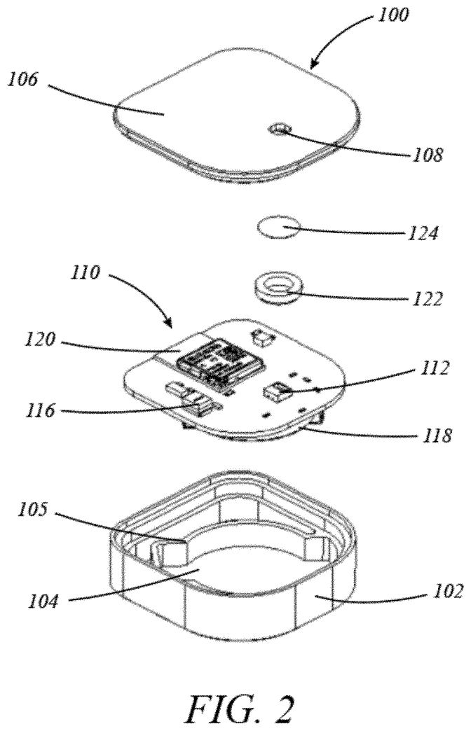

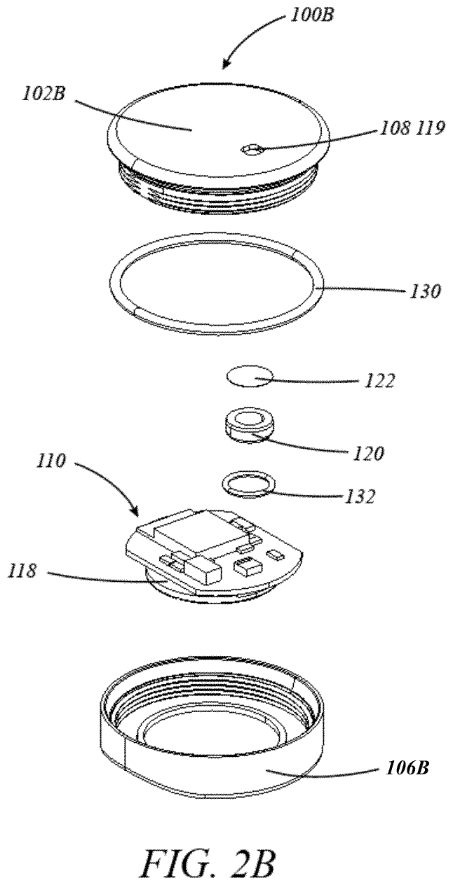

The controller 110 can further comprise a battery 118 which can be capable of powering a solid-state device. The battery 118 may include, for example, a lithium-ion battery, a nickel cadmium battery, an alkaline battery, or the like. In certain embodiments, the battery 118 can be rechargeable by induction circuitry, allowing the user to remotely electrically charge via external induction circuitry. In another embodiment, the battery 118 is rechargeable wherein the wireless module can have a recharging port, allowing the user to electrically charge the battery. In certain embodiments, the housing 102 can include, but is not required to include, a recharging port that can be sealed from the outside environment, allowing the user to selectively access the recharging port to electrically charge the battery 118. In some embodiments, the controller 110 and the battery 118 can be placed into the housing 102 wherein the housing can have one or more slots comprising a battery slot 104, and a controller slot 105 to hold the battery and controller securely within the housing. The housing 102 can be circular, square, triangular, rectangular, or the like in shape having a thickness that can hold the controller 110 and the battery 118. In another embodiment, the housing 102B can be threaded around the inside circumference, and the cover 106B can be threaded around its outside circumference to allow the cover 106B to screw onto and encapsulate a cap O-ring 132 between the housing and the cover as shown in FIG. 2B creating a watertight seal.

In some embodiments, the cover 106 can have a thru hole 108 substantially positioned over the environmental sensor 112 wherein the thru hole allows the environmental sensor access to the outside elements, such as, humidity, temperature, liquids, pressure, gas or the like. The cover 106 can further comprise a bushing 119 located on the bottom side of the cover positioned substantially over the environmental sensor 112. The cover 106 can be circular, square, triangular, or the like in shape. The cover 106 and the housing 102 can be made from, but not limited to, plastics, metals, or other like materials. The wireless module 100 can further comprise a filter element 122, and a locking ring 120 wherein the filter element can be affixed to the cover 106 and can be substantially placed between the cover and the locking ring covering the thru hole. The bushing 119 can be substantially matched to the shape of the locking ring 120 wherein the locking ring can be pushed into the bushing by, for example, interference fit, press fit, loose fit, snap rings, or the like.

In particular embodiments, affixing a filter element 122 to a cover 106 can be defined as press fitting the locking ring 120 into a bushing 119 located on the cover 106 and substantially over the temperature and humidity sensor 112 with the filter element substantially between the locking ring and cover, clamping the filter element to the cover, gluing the filter element to the cover, or the like. The filter element 122 can allow vapor from fluids to pass through to the environmental sensor 112 on the controller 110 without allowing fluids to enter onto and get the controller wet. The filter element 122 can consist of hydrophobic aerogels, polytetrafluoroethylene (“PTFE”), silicone, or the like. The filter element 122 can be circular, square, triangular, rectangular, or the like in shape to completely cover the thru hole 108.

In some embodiments, the cover 106 can be attached to the housing 102 encapsulating the controller 110, battery 118, and bushing 119 wherein the cover can be ultrasonic welded, glued, screwed onto (e.g., as shown in FIG. 2B), rubber sealed, O-rings placed into, gasket sealed or otherwise attached by methods that seal and keep the elements from penetrating and passing through the seal and onto the controller. In another embodiment, a filter O-ring 132 can be placed between the locking ring 120, bushing 108, and the controller 110 creating a barrier between the cover's 102B thru hole 108 as shown in FIG. 2B.

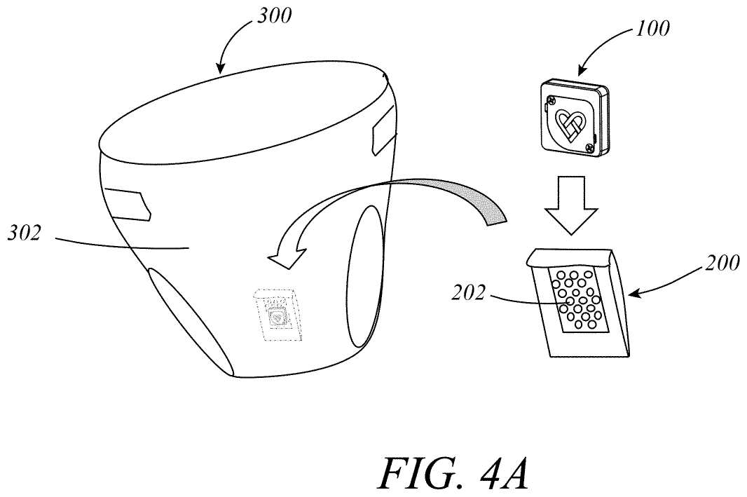

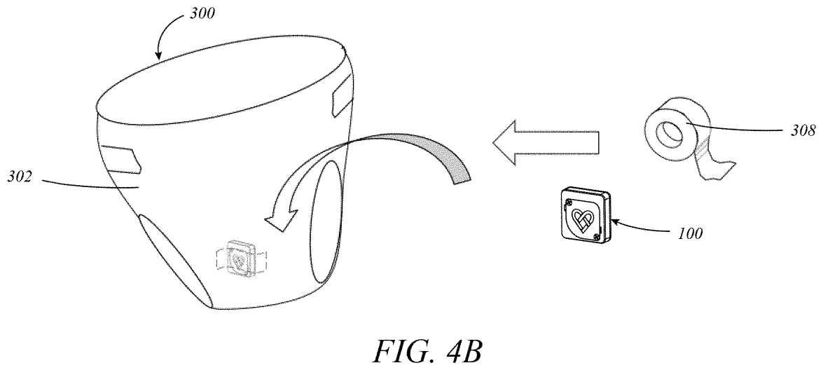

Referring generally to FIG. 4A, which illustrates a wireless module 100 being placed into a secondary pouch 200 and then into an undergarment 300. An undergarment 300 can be defined as, but not limited to, an absorbent article, an incontinence undergarment, a diaper, an article of clothing, shorts, underwear, or the like. In some embodiments, the wireless module 100 can be removably attached using a patch or medical tape to an undergarment 300 or removably placed into a secondary pouch 200 wherein the secondary pouch can be removably attached to an undergarment 300 as shown in FIG. 4B. The secondary pouch 200 can comprise a plurality of thru holes 202 which can be thru either one or both of its surfaces to allow fluids or humidity to enter the inside of the secondary pouch and allow the wireless module to detect a saturation event. The secondary pouch 200 can have an adhesive backing that can be removably attached to the undergarment's surface 302. The secondary pouch 200 can be made out of various materials including, but not limited to, plastics, linens, silk, cotton, or the like, and can be in various shapes such as, but not limited to, circle, square, rectangle, or the like. In another embodiment, a secondary pouch 200 can be omitted, and the wireless module 100 can have an adhesive backing and can be removably attached to the undergarment's surface 302.

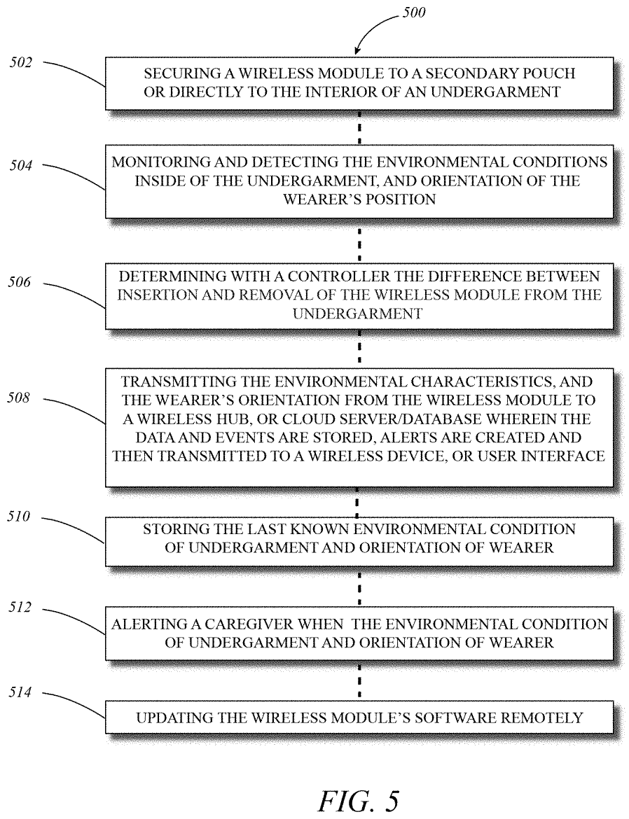

FIG. 5 illustrates a process flow diagram 500 depicting a method for detecting the state of the wireless module 100, in accordance with one, or more embodiments. It is noted herein that the process 500 may be implemented by various embodiments of the wireless module 100. It is further recognized, however, that the process 500 is not limited to the architecture of the wireless module 100. In step 502, a wireless module 100 can be secured to the interior of an undergarment, or in other embodiments secured into a secondary pouch 200 wherein the secondary pouch is attached to the undergarment 300. The secondary pouch 200 can be removable and can be replaced in the undergarment 300 when soiled. In step 504, monitoring and detecting the environmental conditions inside of the undergarment 302 with an environmental sensor 112, and detecting the posture or orientation of the wearer's orientation with at least one motion analyzer 116.

In step 506, transmitting data related to the environmental characteristics and the wearer's posture or orientation from the wireless module to remote device and a hub wherein the data is stored. In some embodiments, a controller 110 can determine the difference between insertion, and removal of the wireless module 100 from the undergarment 302 and then determine the presence or the lack of humidity. For example, the temperature and humidity sensor 112 can detect the humidity characteristics (e.g., via measurement of relative or absolute humidity) of the condition of the undergarment. In some embodiments, the controller 110 can detect the change in temperature when inserted into the undergarment 300, or removed from the undergarment wherein the temperature can be associated with the wearer's body temperature, fluid temperature, and/or ambient temperature when removed. A controller 110 can monitor and detect motion, posture or orientation of the wearer wherein the motion analyzer 116 can detect the motion, posture or orientation characteristic (e.g., x, y, z, positional information of the controller). For example, the controller 110 may detect an angle of orientation of the wearer with respect to the ground, or the surface they are located on, and detect whether there is an acceleration event such as a fall. In certain embodiments, the controller can detect a wearer's fall event by measuring the wearer's static location, and comparing it to an acceleration event detected by the motion analyzer 116.

In step 508, creating alerts based on the data. The wireless module can transmit environmental characteristics, and the wearer's motion, posture or orientation from the wireless module to a wireless hub, or cloud server/database wherein the data and events are stored, alerts are created and then transmitted to a user interface such as a mobile device or web application. At step 510, in some embodiments, the last known environmental condition of the undergarment 300 and motion, posture or orientation of the wearer can be stored wherein the last known condition can be determined, transmitted, and then reset to a dry or static condition. At step 512, creating a display of the environmental conditions or activity or posture or orientation characteristic of the wearer. A caregiver can be alerted through any user interface, such as a display on a mobile device, remote communications device, or a personal computer when the environmental conditions in the wearer's undergarment and/or motion, posture or orientation of the wearer has changed. For example, the wireless module 100 can transmit data collected to a wireless hub wherein the wireless hub relays a string of instructions to the secured cloud which can determine what event has occurred, and then that known event can be relayed to the caregiver. At step 514, a wireless module's 100 can update its software remotely. For example, implementations may include an update or modification of the firmware, programmable hardware, such as by performing a reception of or a transmission of one or more instructions in relation to one or more operations described herein. Alternatively, or additionally, in some variants, an implementation may include special purpose components, or added components. Specifications or other implementations may be transmitted by one or more instances through the wireless hub, or pushed through the secured cloud, through the wireless hub or any other digital device capable of communicating with the wireless module to the wireless module 100.

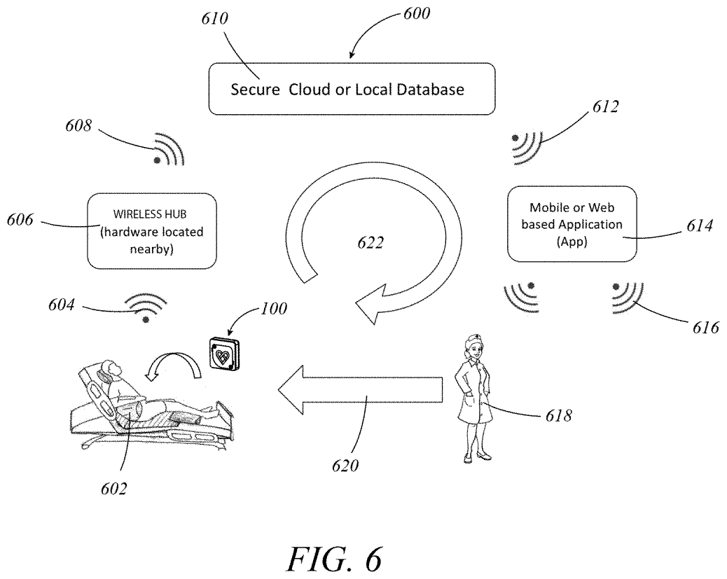

FIG. 6 illustrates a high level schematic view of a method and system for monitoring, detecting and alerting caregivers when humidity or saturation is detected in an undergarment, or the wearer's motion has been detected or when the wearer's posture or orientation has changed. A circle 622 represents a conceptual depiction of the process for monitoring, detecting, and alerting when humidity or saturation is detected in an undergarment using a wireless module 100. A wireless module 100 can be placed and secured into an individual's undergarment 602. The wireless module 100 using a controller 110 having a communications device can transmit a signal 604 to a wireless hub 606 that can be located within the individual's room, common rooms, hallway, caregiver's station, or anywhere within the range of reception of wireless signal 604. In certain embodiments, a wireless hub 606 can be omitted and the wireless module 100 can transmit a signal to a cell phone, a mobile communication device, a LAN, or secured cloud directly. The wireless hub 606 can send a signal 608 to a secured cloud, or local database system 610 wherein the secured cloud or local database system 610 can store the data and events sent by the wireless module. Those having skill in the art will appreciate that there are various vehicles by which a process and/or system and/or technologies described herein can be effected by hardware, software, and/or firmware. The preferred vehicle can vary with the context in which the processes and/or systems and/or other technologies are deployed. For example, if the implementer determines that it is more efficient to bypass a hub and go with a software implementation using Wi-Fi capabilities, or opt for a hardware and/or firmware vehicle, the implementer has that option. Hence, there are several different ways by which the processes and/or device, and/or technologies described herein may be effected, none of which is inherently superior to the other. Other embodiments may include, for example, updates or modifications to software or firmware, or of gate arrays or programmable hardware, such as performing a task to monitor, detect, and then transmit an environmental condition of the undergarment, or motion, posture or orientation of the wearer. In some embodiments, described herein, logic and similar implementations may include software or other control structures. Electronic circuitry, for example, may have one or more paths of electrical current constructed and arranged to implement various functions as described herein.

In some embodiments, the secured cloud system 610 can create alerts which then communicate 612 to user interface, such as a mobile or web based application (App) 614 using a computing device wherein information is communicated wirelessly via for example, but not limited to, a BLUETOOTH Low Energy (BLE), BLUETOOTH module, Wi-Fi module, IEEE 802.15.4, Z-Wave, Single/Dual Mode Radio Chip, cellular networks, or the like on a local cloud server 610 to remote computing devices 614, wherein the alerts and graphical representations of the historical data, and events are displayed to a caregiver's 618 cellular phone, computer, tablet, or the like 616 wherein a caregiver can be for example, but not limited to, nurse, parent, caregiver, nanny, babysitter, or the like. Information transmitted to the caregiver can include the identity of the user of the wireless module 100, the location of the user, other information from the user's profile, the nature of the event, or time elapsed since the event.

In certain embodiments, the wireless module 100 is designed to recognize when an undergarment is wet or saturated. When the wireless module 100 can no longer communicate with the wireless hub 606 the caregiver 618 receives a notification that the wireless module is either out of range, the patient has moved, or the wireless module is out of power and the batteries need to be recharged or replaced. In a general sense, those that are skilled in the art will recognize that the various aspects described herein can be implemented individually, or as any combination thereof can be viewed as being composed as a “controller” which includes electrical circuitry. The control, or electrical circuitry includes, but not limited to having at least one discrete electrical circuitry, electrical circuitry having at least one discrete electrical circuit, electrical circuitry having at least one application specific integrated circuit, electrical circuitry forming a general-purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, Wireless LAN, Wireless HUB, optical-electrical equipment, etc.). Those having skill in the art will recognize that the subject matter described herein may be implemented in an analog or digital or some combination thereof

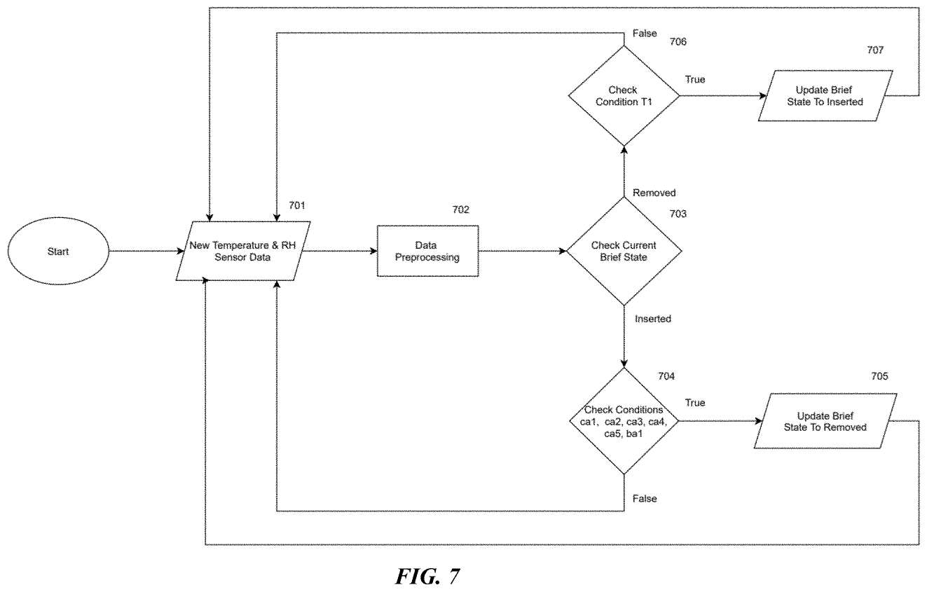

Referring FIG. 7 illustrates a process flow diagram for depicting a method or system for detecting automatic removal of a wet or saturated undergarment and insertion of a clean undergarment in replacement and counting the number of undergarment changes performed daily by the caregivers. At 701 a wireless module can be powered by a rechargeable or replaceable battery wherein the battery can be such as, for example, lithium ion, nickel cadmium, nickel metal hydride, or the like. The processing unit initializes the software and peripherals and then it performs a sensor relative humidity (“RH”) and temperature measurements at a fixed frequency. At step 702, data is being pre-processed wherein the appropriate feature vectors can be used to determine the brief insertion state transitions. Seven aggregating functions are accumulating information regarding the temperature and relative humidity changes over time by employing rolling window aggregation and differentiation. At step 703, based on the current brief insertion state the logic branches into its respective flow. Here, the current brief insertion state variable is read and a logical branching operation is performed.

In certain embodiments, at step 704, conditions for ca1, ca2, ca3, ca4, ca5, ba1 are checked wherein there can be some aggregated features that can be evaluated with a logical function to determine a brief insertion state. Features ca1, ca2, ca3, ca4 encode information about the variation in temperature features for various thresholds, while features ca5 and ba1 are being used to encode information about variation in relative humidity features. The logical function that combines these features is then able to generate a Boolean value about the brief insertion state. If the value is false, then the brief insertion state is unchanged. If the value is true, then an additional check is performed.

At step 705, the current brief insertion state can be switched into the Removed state and can store this information into the brief insertion state variable. While the brief insertion state is set to Removed, the sensor state is considered to be off the body.

At step 706, this step is triggered if the current brief insertion state is set to Removed and the condition T1 is evaluated. The condition T1 encodes changes in the temperature readings through a rolling window aggregation. If the condition is not met, then the insertion state remains unchanged. If the condition is met, then the process to change the brief insertion state to Inserted is triggered. At step 707, the current brief insertion state is switched to the Inserted state and stores this information into the brief insertion state variable. While the brief insertion state is set to Inserted the sensor is considered to be on the body.

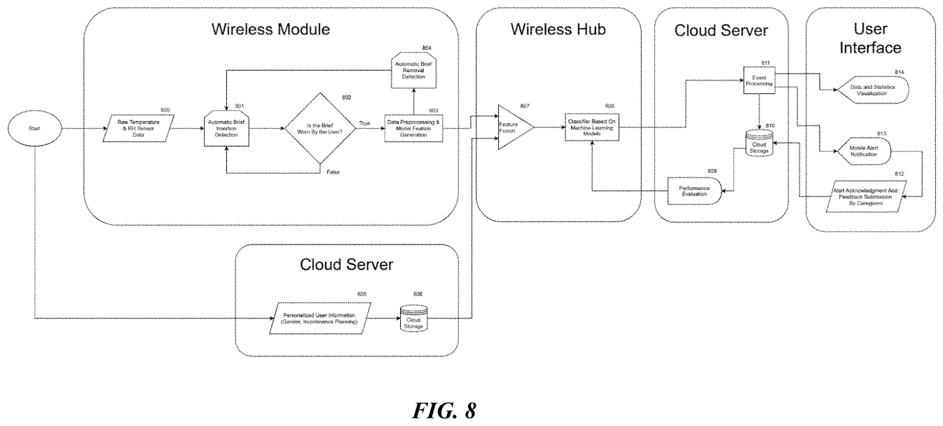

Referring to FIG. 8, a process flow diagram for depicting a method or system for automatic monitoring of the wetness of an undergarment wherein at step 800 a raw temperature and RH sensor data is detected. The wireless module can be powered by a rechargeable or replaceable battery. The processing unit initializes the software and peripherals and performs a sensor relative humidity (RH) and temperature measurements at a fixed frequency. At step 801, the temperature vector created using the measurements from step 800 can be aggregated into a new vector with the application of a mathematical rolling average function. This vector is then translated into the protective brief insertion state variable by employing thresholding logic. At step 802, detects if the brief is worn by the user wherein the algorithm flow switches based on the brief insertion state variable value as it was described in step 801. If the brief insertion state is set to the False value, then the algorithm remains in the same state and awaits new data. If the brief insertion state is set to True, then the algorithm switches to an active state which begins the execution of subsequent processes.

At step 803, data processing and model feature generation wherein a new feature vector is calculated by aggregating the relative humidity vector. This vector is responsible for detecting anomalies in the data due to brief insertion transient phenomena. By detecting the variation in the readings and taking into account temporal information about the brief insertion state this process is able to determine the optimal starting point of the model feature creation. It is important for subsequent steps to receive clean data therefore this step ensures this. At step 804, once the transient phenomena as described by step 803 have receded the main pre-processing step starts executing. Empty vectors that will be populated by the model features are being allocated in the memory medium. New raw features that are the mathematical output of the combination between temperature and relative humidity features are being generated and stored in new vectors. New aggregator functions are being initialized with values as received from step 803. The necessary conditions for resetting the execution of the algorithm can be generated. For each of those vectors specific fixed thresholds are set that are used to convert the continuous values into logical variables that are being evaluated with logical operations and are used to automatically detect the brief removal from the user. If the outcome of the logical operations results in True, then a brief removal event is generated. When such an event is detected, the execution of the wetness or saturation detection algorithm is halted, and its current state is being reset to the initial step. The aggregator functions are being emptied in order to be re-initialized again on the next brief insertion. After initialization as described in steps 803 and 804 is successful the aggregator functions start accumulating the incoming data in a state-full manner. The role of these aggregating functions is to efficiently extract different sets of information from the input data with the use of mathematical transformations. Temporal dependencies between each of the aggregating functions are encoded in a time-invariant format which ensures forward information flow on subsequent algorithm components which are operating in a stateless manner. The output of each aggregating function together with brief removal and insertion events are then then transmitted to the wireless hub or any other computer device that implements functions described in steps 807 and 808.

At step 805, the personalization of user information i.e., gender and brief type utilisation planning can be inputted into the system. Additional algorithm sensitivity adjustment factor can also be entered by the caregiver. The information is being interfaced with the system through a specialized caregiver user interface, specifically designed for this role. Information being gathered corresponds to the gender and brief type utilisation planning of the user. At step 806, the information can be gathered and can be encoded and stored into a secure cloud server in an anonymous manner to ensure the full protection of the user and compliance to existing regulations regarding user data privacy. This information is being forwarded as a column vector of size (N, 1), with N being the total number of features that will serve as a personalization context vector to the model features as they are generated from the wireless module. This vector is being sent to the wireless hub or any computer that implements functions 807 and 808.

At step 807, feature fusion is performed wherein the wireless hub device receives the data from the wireless module. By having both the model features in a vector format and then context feature vector, the hub device concatenates both vectors into a single column vector of size (16+N, 1) to be used for brief wetness state estimation. At step 808, the concatenated feature vector is used as input to a machine learning model to predict the current brief wetness state based on both the sensor readings and the personalized information. The machine learning model can be trained on a carefully curated collection of features generated from the wireless module and personalized information as it is collected and stored in steps 805 and 806 in a supervised manner. The labels for the supervised learning process have been gathered from caregiver feedback during trial sessions as they will be described in steps 812 and 810. The training is performed in-house and off-line with equipment separate from the distributed architecture. The training accuracy can be cross validated using evaluation data not present on the training set.

The model is generating a continuous prediction value depicting the wetness state of the brief in terms of saturation after performing inference on the input data. The prediction value of the model is received, as described in step 808, and then a thresholding operation is applied to convert the continuous value into discrete brief wetness state categories. This operation is further enhanced from live feedback received by the caregiver in step 812 and the optimal operating state is being re-evaluated and tuned on a fixed time interval or continuously. The system can adapt over time to new users achieving higher performance than its baseline performance during off-line training. The brief wetness state is received and evaluated whether it is below or above a saturation threshold. If the current brief wetness state is below the saturation threshold then no further action is taken. If the current brief wetness state is above the saturation threshold then a subsequent process is being triggered. If the brief saturation state is determined to be saturated, then a saturation event is generated with the current timestamp. This event, together with the relevant meta-data, including the brief change events are transmitted from the hub device to the cloud or local server.

At step 811, the cloud server receives all events from all wireless hub devices and initially performs a filtering logic to remove redundant events based on the event timestamp and even type. This step then forwards the events to the cloud storage, and the newly created events to subsequent processes. At step 810, the filtered events together with relevant meta-data and the accompanying anonymous user information are stored in the database. This database retains user or caregiver submitted feedback including but not limited to algorithm performance evaluation. The information is securely stored in order to comply with regulatory requirements about user data privacy. At step 811, newly created saturation events can be transformed into alerts in order to notify the caregiver of the current brief wetness or saturation state. Notifications can be then pushed to the specific devices that host the caregiver user interface. Step 809 can be triggered on a fixed time interval or by appearance of new useful data generated by step 810. In the step 809 performance metrics as they are described in step 810 are being gathered in order to calculate the current performance of each active user automatically. A fixed rolling window can be used to fetch historical performance information up to a certain past epoch. The performance can be measured in terms of sensitivity, specificity and area under the RoC curve. For each active user a new optimal operating point can be generated automatically and this information can be passed down to the predictive model as described in step 808.

At step 813, the notifications as described in step 811 are being pushed to the caregiver's user interface to notify the caregiver of the saturated brief state. Along with the notification, the user interface displays relevant meta-data about the user. At step 812, the caregiver is able to submit feedback about the performance of the algorithm as well as additional information about the user and brief wetness state from a predefined form of communication. This information is passed to the cloud server as described in step 810, so that the algorithm can self-improve and increase its performance, as well as adapt to each user's specific needs. At step 814, the aggregated data as generated, as discussed above, are being visualized in the user interface so that the caregiver can keep track of past history regarding saturation alerts, brief changes and any other information or events generated by the wireless module, wireless hub or cloud or local server. Aggregated statistics such as the average number of changes per flexible time intervals may also be available.

It is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

What is claimed is:

1. A system for detecting an environment having at least one environmental feature, the system comprising:
a wireless module comprising a controller accommodated within a housing at least partially separating the controller from the environment, at least one environmental sensor at least partially in contact with the environment to detect at least one of the environmental features, and a motion sensor to detect motion, posture or orientation of a wearer of the wireless module.

2. The system according to claim 1, wherein the system further comprises:
a controller within the housing wherein the controller comprises a first transceiver and a processing unit and the first transceiver is coupled to the processing unit;
wherein the processing unit stores data related to changes in the environment and/or motion, posture or orientation data and wherein the processing unit executes a first part of a distributed algorithm and transmits information via the first transceiver to a second transceiver inside a wireless hub or a cellular base station where the information is used in the execution of a second part of the distributed algorithm resulting in the detection of a wet or saturated undergarment and of the posture or orientation of the wearer of the wireless module.

3. The system according to claim 1, wherein the controller comprises a transceiver, a processing unit and a memory medium wherein the controller is suitable for executing programmed instructions stored on the memory medium, wherein program instructions are configured to cause the transceiver to wirelessly transmit information to a remote device about at least one environmental feature and the motion, posture or orientation of the wearer of the wireless module.

4. The system according to claim 1, wherein at least one environmental feature is selected from the group comprising humidity, temperature, and the presence of a compound.

5. The system according to claim 1, wherein the environmental sensor is within the housing and wherein the housing has at least one thru hole substantially positioned over the environmental sensors, wherein the wireless module further comprises a bushing substantially positioned between the environmental sensors and the housing.

6. The system according to claim 4, wherein the wireless module further comprises a filter element, and a locking ring wherein the filter element is affixed into the bushing and the locking ring secures the filter element substantially between the housing, and the locking ring.

7. The system according to claim 1, wherein the housing comprises a cover encapsulating the controller and a power supply wherein the wireless module is resistant to fluids.

8. The system according to claim 1, wherein the wireless module is removably attached to an undergarment using a patch or medical tape or is secured into a secondary pouch wherein the secondary pouch can be removably attached to an undergarment, wherein the secondary pouch has a plurality of holes on its surface to allow fluids to penetrate the secondary pouch.

9. The system according to claim 1, further comprising a battery, wherein the battery is rechargeable or replaceable and wherein the controller is configured to save power during operation by intermittently measuring various characteristics and processing data.

10. The system according to claim 2, wherein the remote device is a cell phone or a mobile communications device or a wireless communication computer.

11. The system according to claim 1, wherein the motion sensor is at least one of an accelerometer or a gyroscope or a magnetometer or a barometer.

12. The system according to claim 2, wherein the program instructions are further configured to compare the state of the undergarment to determine whether there is an environmental change with the environmental sensors, and then transmit that state to a wireless hub.

13. A method of detecting wetness or saturation and motion, posture or orientation of an undergarment comprising:
securing a wireless module to a secondary pouch or directly to an interior of an undergarment;
monitoring environmental conditions inside of the undergarment with an environmental sensor, and an orientation characteristic of a wearer's position with at least one motion sensor;
transmitting data related to the environmental characteristics and the wearer's posture or orientation from the wireless module to remote device or wireless hub wherein the data is processed and then events and data are communicated to a local or cloud server creating alerts based on the events and data; and;
alerting a caregiver via a communications device when the environmental conditions and posture or orientation of the wearer has changed or motion of the wearer has been detected.

14. The method of claim 12, further comprising storing a last known environmental condition, or posture or orientation characteristic and creating an alert based on a change in the environmental condition or posture or orientation characteristic.

15. The method of claim 12, further comprising:
creating a display of the environmental condition or activity or posture or orientation characteristic of the wearer.

16. The method of claim 12, wherein an alert is created when the wearer rapidly changes from a standing or sitting to an at least partially prone orientation.

17. The method of claim 12, wherein the caregiver is alerted when the undergarment changes from dry to wet or saturated.

18. A method of claim 12, wherein the mobile communications device comprises at least one, smartphone, personal digital assistant, a tablet device, a table PC, a laptop, a smart book, or an ultra-book.

19. The system according to claim 2, wherein the processing unit executes a first part of a distributed algorithm and transmits information via the first transceiver to a second transceiver inside a wireless hub or a cellular base station where the information is used in the execution of a second part of the distributed algorithm resulting in the automatic detection of removal of a wet or saturated undergarment and insertion of a clean undergarment in replacement and may as a result count the number of undergarment changes performed daily by the caregivers thus providing reliable and traceable information on the quality of the incontinence care.
